(19) United States
(12) Patent Application Publication
(10) Pub. No.: US 2011/0158912 A1
(43) Pub. Date: Jun. 30, 2011

(54) METHOD AND DEVICE FOR DETERMINING THE SEVERITY OF A PAIN DISORDER

(76) Inventors: Tony Wright, Western Australia (AU); Penny Moss, Western Australia (AU); Heather Benson, Western Australia (AU)

(21) Appl. No.: 13/057,836

(22) PCT Filed: Aug. 12, 2009
(86) PCT No.: PCT/AU2009/001037

§ 371 Date: Feb. 7, 2011

(30) Foreign Application Priority Data
Aug. 13, 2008 (AU) 2008904136

(51) Int. Cl. A61K 49/00

(52) U.S. Cl. 424/9.2; 424/9.1

(57) ABSTRACT

A method for determining the severity of a pain disorder in a subject, comprising the steps of: (i) topically applying an effective amount of menthol or a functional equivalent thereof to the subject's painful area and other locations; and (ii) measuring the response of the subject

### FIELD OF THE INVENTION

The present invention relates to a prognostic method, device and kit for determining the development of a chronic pain disorder in a subject. The present invention also relates to a method, device and kit for determining the severity of a pain disorder. More particularly, the present invention relates to a method, device and kit for determining the severity of a painful musculoskeletal condition.

### BACKGROUND ART

Pain disorders are complex, disabling and often chronic. Although initially the result of localised tissue injury, chronic pain states persist beyond resolution of tissue damage.

Many musculoskeletal conditions and injuries develop into chronic pain disorders. It is a common and widespread problem that creates a large financial burden. A US study in 1995 estimated the direct cost of chronic musculoskeletal conditions amounted to US$88.7 billion and the indirect costs amounted to US$126.2 billion (WHO Technical Report Series 919 “The Burden of Musculoskeletal Conditions at the Start of the New Millennium” World Health Organisation 2003 ed; Whitehouse Station, N.J.).

Particular groups of patients with chronic musculoskeletal conditions require large amounts of costly health care input and other financial support. For example, the FIA Foundation (a UK-based road safety organisation) has calculated the cost of whiplash associated disorders to be 8 billion in Europe and up to $US 2.7 billion in North America. Osteoarthritis is currently the tenth most frequently managed problem by GPs in Australia (Victorian Government statistics). In the US, the direct health costs of arthritis amount to $US 20 billion, with additional lost wages calculated as a further $60 billion (WHO Technical Report 2003). Those with more pain tend to require a disproportionate percentage of health care resources.

There is no simple and reliable means to predict the likely development of a chronic musculoskeletal pain disorder at an early stage in the condition. Consequently, these patients are often not recognized until their condition has already consumed significant health care dollars. A simple and reliable prognostic indicator for chronic musculoskeletal conditions would enable early identification and initiation of intervention with these patients.

Abnormal cold sensitivity is an indicator of poor clinical outcomes and development of chronicity in subjects with musculoskeletal pain disorders such as whiplash. At present, cold sensitivity is assessed using a thermode, a relatively expensive piece of machinery that produces varying thermal stimuli. However, this equipment is not readily available outside of hospitals and specialist clinics.

The present invention seeks to at least partially overcome the above deficiencies in the prior art.

### SUMMARY OF THE INVENTION

The inventors have surprisingly discovered that, by applying menthol to a subject and then measuring the subject's response to the menthol application, they are able to predict a subject's susceptibility to the development and severity of pain disorders.

In a first aspect, the invention is a method for determining the severity of a pain disorder in a subject, comprising the steps of:
(i) topically applying an effective amount of menthol or a functional equivalent thereof to the subject's painful area and other locations; and
(ii) measuring the response of the subject.

In an embodiment of the first aspect of the invention, the invention is a method for determining the severity of a pain disorder in a subject, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder.

The present invention has particular benefit in the determination or identification of subjects that may be suffering from a chronic pain disorder or who are likely to suffer from such an ailment.

In a second aspect, the invention is a method for identifying a chronic pain disorder in a subject comprising the steps of:
(i) topically applying an effective amount of menthol or a functional equivalent thereof to the subject's painful area and other locations; and
(ii) measuring the response of the subject.

In an embodiment of the second aspect of the invention, the invention is a method for identifying a chronic pain disorder in a subject, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject has a chronic pain disorder, wherein the standard differentiates abnormal menthol responses from normal menthol responses and abnormal menthol responses are indicative of chronic pain in a subject.

Those of skill in the field of pain management will recognise that this invention has value beyond identifying a subject's state of pain at a particular time point. The invention may also be employed to monitor the progression of a pain disorder by assessing the severity of the pain over a time course.

In a third aspect, the invention is a method for assessing the severity of a pain disorder over time comprising the repeated steps of:
(i) topically applying an effective amount of menthol or a functional equivalent thereof to the subject's painful area and other locations at least twice over a specific period of time; and
(ii) measuring and comparing the responses of the subject from each application.

In an embodiment of the third aspect of the invention, the invention is a method for assessing the severity of a pain disorder over time, comprising the steps of:
(i) topically applying a composition comprising 5-50% menthol or a functional equivalent thereof to the subject;
(ii) identifying the subject's response to the composition;
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder; and
(iv) repeating steps (i) to (iii) at periodic intervals to develop a time course profile of the subject's pain disorder.

The breadth of applications to which the invention may be put also extends to use in the assessing the efficacy of a pain treatment.

In a fourth aspect, the invention is a method for assessing the efficacy of a treatment for a subject's pain disorder, comprising the steps of:
(i) topically applying an effective amount of menthol or a functional equivalent thereof to the subject's painful area and other locations before and after the treatment; and
(ii) measuring and comparing the responses obtained from each application.

In an embodiment of the fourth aspect of the invention, the invention is a method for assessing the efficacy of treatment of a subject's pain disorder, comprising the steps of:
(i) topically applying a composition comprising 5-50% menthol or a functional equivalent thereof to the subject at two different time points, wherein at least one of the selected time points is after the subject receives treatment for the pain disorder;
(ii) identifying the subject's response to the composition at the different time points;
(iii) comparing the subject's response at each time point to a pre-determined set of standard responses to determine the subject's pain response, wherein the standard differentiates abnormal menthol responses from normal menthol responses and abnormal menthol responses are indicative of the development of a pain disorder.

In a fifth aspect of the invention, the invention is a method for developing or tailoring a treatment regime to treat a subject's pain disorder, comprising the steps of:
(i) topically applying a composition comprising 5-50% menthol or a functional equivalent thereof to the subject at two different time points, wherein at least one of the selected time points is after the subject receives treatment for the pain disorder;
(ii) identifying the subject's response to the composition at the different time points;
(iii) comparing the subject's response at each time point to a pre-determined set of standard responses to determine the subject's pain threshold, wherein the standard differentiates abnormal menthol responses from normal menthol responses and abnormal menthol responses are indicative of the development of a pain disorder.

Ideally, the fourth and fifth aspects of the invention are repeated during the course of a subject's treatment for a pain disorder. Such ongoing measurement will provide a time course measure of the subject's pain threshold which can then be related back to the treatment employed in the subject.

A sixth aspect of the invention provides a method for screening a subject to determine if the subject is susceptible to the development of chronic pain if that subject is stricken with a pain disorder, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder.

In a seventh aspect, the invention is a kit comprising:
(i) a dispensing means for topically delivering an effective amount of menthol or a functional equivalent thereof to a subject with a pain disorder, and
(ii) a means for the quantitative and/or qualitative measurement of the subject's response and interpretation thereof.

According to the seventh aspect of the invention, the dispensing means will deliver an amount of menthol of between 10 and 50% w/w. Preferably, the menthol will be delivered in a formula or composition comprising other constituents. Those other constituents desirably will not interact in an overly adverse manner with the menthol.

The present invention provides a composition adapted for topical administration to a subject comprising an effective amount of menthol or a functional equivalent thereof.

The present invention also provides a dispensing means for topically delivering an effective amount of menthol or a functional equivalent thereof to a subject with a pain disorder, the dispensing means comprising the effective amount of menthol or a functional equivalent thereof. The dispensing means may also comprise a controlled release means.

### BRIEF DESCRIPTION OF THE DRAWINGS

FIGS. 1 to 5 show a number of examples of means for the quantitative and/or qualitative measurement of the subject's response and interpretation thereof that could be used in a method or kit of the present invention.

FIG. 6 is a schematic cross sectional view of a dispensing means according to a first embodiment of the present invention;

FIG. 7 is a schematic cross sectional view of a dispensing means according to a second embodiment of the present invention; and

FIG. 8 shows the Cold Pain Sensitivity at knee, ankle and elbow test sites, comparing a subject with osteoarthritis to healthy control subjects.

### DETAILED DESCRIPTION OF THE INVENTION

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document or reference cited in this text is expressly incorporated herein in its entirety by reference, which means that it should be read and considered by the reader as part of this text. That the document or reference cited in this text is not repeated in this text is merely for reasons of conciseness. None of the cited material or the information contained in that material should, however be understood to be common general knowledge.

The present invention is not to be limited in scope by any of the specific embodiments described herein. These embodiments are intended for the purpose of exemplification only. Functionally equivalent products, formulations and methods are within the scope of the invention as described herein.

The invention described herein includes one or more range of values. A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### Method of Determining the Likelihood of a Chronic Pain Disorder

In most normal subjects, a cold stimulus below a minimum threshold will elicit a sensation of pain. A major characteristic of sufferers of chronic pain is that they find that normally innocuous cool stimuli above the threshold will produce pain (cold allodynia) and/or they feel a heightened sensitivity towards painful cold stimuli (cold hyperalgesia).

Whilst not wishing to be bound by any particular mode of action, the inventors believe that a subject with a nociceptive system that is sensitised to neuronal inputs, for example from the TRPM8 cold-menthol receptor (probably through a centrally-controlled mechanism), has a greater than average likelihood of developing a severe chronic pain disorder.

By measuring a subject's sensitivity towards cold stimulus, one can determine whether a subject has increased risk of developing long term issues with pain from, for example, an injury or other pain disorder. A subject with increased sensitivity towards cold may be at increased risk of suffering from a pain disorder that is more severe, difficult to treat and/or causes longer term problems and suffering.

As used herein “increased sensitivity to cold” refers to a subject being more sensitive to pain caused by a cold signal than normal control subjects. Thus, a cold temperature which would not generate a pain signal in a normal subject may cause a pain signal in a subject which may be indicative of the likelihood of that subject suffering a chronic pain disorder.

It is believed that menthol and functional equivalents thereof trigger the same neuronal pathways as cold signals. Therefore, subjects with increased sensitivity to cold will also have increased sensitivity to menthol or a functional equivalent thereof. The invention uses this sensitivity to provide a simple measure for determining the likelihood that a subject is at risk of suffering from a chronic pain disorder. It is proposed that, if a subject has an increased sensitivity to menthol or a functional equivalent thereof, they will also be at increased risk of developing a severe chronic pain disorder.

In a normal control subject, application of a given dose of menthol may generate a cold sensation and low or no pain response. However, the same dose of menthol applied to the skin of a subject at increased risk of developing a chronic pain disorder may generate a high level of pain and a strong response, such as a stinging, painful, tingling or severe cold sensation. Alternatively, the applied dose may cause a paradoxical response, wherein the subject feels a burning or heat sensation, rather than the cold sensation usually triggered by application of menthol.

In a first aspect, the invention is a method for determining the severity of a pain disorder in a subject, comprising the steps of:
(i) topically applying an effective amount of menthol or a functional equivalent thereof to the subject's painful area and other locations; and
(ii) measuring the response of the subject.

In an embodiment of the first aspect of the invention, the invention is a method for determining the severity of a pain disorder in a subject, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder.

Preferably, the normal menthol response is determined by topically applying a composition comprising 5-50% menthol or a functional equivalent thereof to a cohort of normal individuals and determining the sensitivity of the cohort to the composition; and the abnormal menthol response is determined by topically applying a composition comprising 5-50% menthol or a functional equivalent thereof to a cohort of individuals suffering from a chronic pain disorder who have an abnormal pain response and determining the sensitivity of the cohort to the composition.

In a second aspect, the invention is a method for identifying a chronic pain disorder in a subject comprising the steps of:
(i) topically applying an effective amount of menthol or a functional equivalent thereof to the subject's painful area and other locations; and
(ii) measuring the response of the subject.

In an embodiment of the second aspect of the invention, the invention is a method for identifying a chronic pain disorder in a subject, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject has a chronic pain disorder, wherein the standard differentiates abnormal menthol responses from normal menthol responses and abnormal menthol responses are indicative of chronic pain in a subject.

There are many means of measuring the response caused by a given stimulus, such as by the complete McGill Pain Questionnaire or the Modified McGill Pain Questionnaire (short form); the Visual Analog Scale (VAS); the Pain Quality Assessment Scale; the Neuropathic Pain Scale; a Simple Descriptive Score (SDS); a pictorial scale, such as the Wong-Baker Faces Pain Rating Scale (FRS) (Baker-Lefkowicz et al., 1996); a Numerical Rating Scale (NRS) etc. A person skilled in the art will know a range of suitable methods which can be used to measure the response generated by the topical application of a dose of menthol or functional equivalent.

Preferably, the nature of the subject's response to the menthol is identified using a descriptor-based assessment method, wherein the subject rates their response to topically applied menthol by the qualitative choice of words which describe the sensation that they are feeling. This description based assessment is then preferably converted into a numerical score which can be compared to scores generated from other subjects, for example a cohort of normal individuals and a cohort of individuals suffering from chronic pain disorders.

Thus the invention provides a method for identifying a chronic pain disorder in a subject, comprising the steps of:
(i) topically applying a composition comprising 5-50% menthol or a functional equivalent thereof to the subject;
(ii) identifying descriptor words which describe the subject's response to the composition;
(iii) converting the description of the response into a value that rates the response; and
(iv) comparing the subject's response to a pre-determined set of standard responses to determine whether the subject has a chronic pain disorder, wherein the standard differentiates abnormal menthol responses from normal menthol responses and abnormal menthol responses are indicative of chronic pain in a subject.

For example, the subject's response may be identified using the complete McGill Pain Questionnaire or the Modified McGill Pain Questionnaire (short form); the Pain Quality Assessment Scale; or the Neuropathic Pain Scale.

The invention further provides a method for identifying a chronic pain disorder in a subject, comprising the steps of:
(i) topically applying a composition comprising 5-50% menthol or a functional equivalent thereof to the subject;
(ii) identifying the subject's response to the composition using a numerical scale to rate the intensity of the subject's response; and
(iii) comparing the subject's response to a pre-determined set of standard responses to determine whether the subject has a chronic pain disorder, wherein the standard differentiates abnormal menthol responses from normal menthol responses and abnormal menthol responses are indicative of chronic pain in a subject.

For example, the subject's response may be identified using the Visual Analog Scale (VAS); a pictorial scale, such as the Wong-Baker Faces Pain Rating Scale (FRS); or a Numerical Rating Scale (NRS).

Preferably, the invention provides a method for identifying a chronic pain disorder in a subject, comprising the steps of:
(i) topically applying a composition comprising 5-50% menthol or a functional equivalent thereof to the subject;
(ii) identifying descriptor words which describe the subject's response to the composition;
(iii) converting the description of the response into a value that rates the response;
(iv) identifying the subject's response to the composition using a numerical scale to rate the intensity of the subject's response;
(v) combining the values from (iii) and (iv); and
(vi) comparing the subject's response to a pre-determined set of standard responses to determine whether the subject has a chronic pain disorder, wherein the standard differentiates abnormal menthol responses from normal menthol responses and abnormal menthol responses are indicative of chronic pain in a subject.

In a most preferred aspect, the pre-determined set of responses is generated using both the descriptor and numerical scale. The identification of the subject's response may be carried out using either the descriptor or the numerical scale. However, in order to increase the sensitivity and accuracy of the method, it is preferred that the subject's response be determined using a combination of both the descriptor and the numerical scale.

The present invention, however, also provides a specific method of assessing and measuring the response generated by the topical application of a given dose of menthol or a functional equivalent thereof. This method is provided as an illustration of the specific utility of the Applicant's invention and should not be regarded as limiting on the breadth of the disclosure herein, as the methodology described is broader than that now being illustrated. This method is herein referred to as the Cold Pain score. Preferably, a subject's response to the menthol is measured using this score.

The Cold Pain score may be determined by the following method:
(a) topically applying a composition comprising 5-50% menthol or a functional equivalent thereof to the subject;
(b) rating the subject's response to the menthol or functional equivalent thereof according to a description of the sensation by selecting as many or as few of the words in Table 1 to describe the sensation(s) that they are feeling;
(c) converting the words selected in part (b) into a Descriptor rating; and
(d) determining a Cold Pain score from the Descriptor rating generated in part (c).

Alternatively, the Cold Pain score may be determined by the following method:
(a) topically applying a composition comprising 5-50% menthol or a functional equivalent thereof to the subject;
(b) rating the intensity of the subject's cold sensation, heat sensation and pain sensation generated in response to the menthol or functional equivalent thereof on a scale;
(c) converting the rating from part (b) into an Intensity rating; and
(d) determining a Cold Pain score from the Intensity rating generated in part (c).

Preferably, the Cold Pain score is determined by the following method:
(a) topically applying a composition comprising 5-50% menthol or a functional equivalent thereof to the subject;
(b) rating the subject's response to the menthol or functional equivalent thereof according to a description of the sensation by selecting as many or as few of the words in Table 1 to describe the sensation(s) that they are feeling;
(c) converting the words selected in part (b) into a Descriptor rating;
(d) rating the intensity of the subject's cold sensation, heat sensation and pain sensation generated in response to the menthol or functional equivalent thereof on a scale from 1 to 10;
(e) converting the rating from part (d) into an Intensity rating; and
(f) determining a Cold Pain score from the ratings generated in parts (c) and (e).

Descriptor Rating

TABLE 1
Cool, cold, icey, freezing, warm, hot, burning, numb, tingling, itchy,
prickling, stinging, dull, sharp, penetrating, intense, aching,
pulsing/throbbing, spreading

To determine the Descriptor rating, the words the subject has select from Table 1 are compared to Table 2 and given a Descriptor score depending on in which column it occurs (e.g. the word “penetrating” would be given a Descriptor score of 3). The Descriptor scores are then added up and divided by the total number of words chosen to give the final Descriptor rating.

TABLE 2
Descriptor score categories

| 1 | 2 | 3 | 4 | 5 |
|---|---|---|---|---|
| dull | cold | penetrating | hot | burning |
| cool | tingling | itchy | prickling | stinging |
| numb | pulsing/throb | spreading | freezing | |
| | sharp | warm | intense | |
| | | Icey | aching | |

Thus, for example, Subject A first chooses the words “freezing”, “intense” and “burning” from Table 1. These words are then compared to Table 2, where “freezing” and “intense” score 4 each and “burning” scores 5. These numbers are then tallied to give a Descriptor score of 13, which is then divided by 3 (the total number of words chosen) to give a final Descriptor rating of 4.3.

The actual words provided in Table 1 may be altered or varied from those shown above. This may be of particular relevance if translating the method of the present invention into languages other than English or if using the method in relation to various cultural groups. It has been well documented that there are cultural differences between the useful words which can be used in descriptor methods of determining pain. For example, the McGill Pain Questionnaire has been translated in a large number of languages, with the words selected to fill the word tables not being exact translations of the original English words, but rather being culturally appropriate words. Similar translational modifications may be employed in the present method, provided that the words chosen allow the distinguishing of an abnormal menthol response from a normal menthol response. Various pain descriptor scales are known and may be referred to for alternative descriptor words to fill the table, for example the Pain Quality Assessment Scale or the Neuropathic Pain Scale.

Preferably, the words chosen to fill the column on the table which provides at least the highest Descriptor score (e.g. column 5 in Table 1 above) are very similar in nature to the English words shown above. Thus, there must be a high determined correlation between the words chosen and an abnormal pain response. Preferably, the words chosen to fill the column with the highest Descriptor score are words which reflect that the sensation felt by the subject is a burning and/or stinging sensation.

The words used to fill the columns may also be moved from column to column, changing their Descriptor Score. Thus, for example, the word “tingling” which is currently in column 2 of Table 1 may be moved to column 1, thus reducing the Descriptor score of this word from 2 to 1. Other such modifications are also contemplated, so long as the overall composition of the table and the words therein still permits the distinguishing of an abnormal menthol response from a normal menthol response.

Preferably, the words chosen to fill the column on the table which provides the highest Descriptor score (i.e. column 5) remain at least the two words shown above, “burning” and “stinging”. However, other words may be moved from, for example, column 4 into column 5.

Intensity Rating

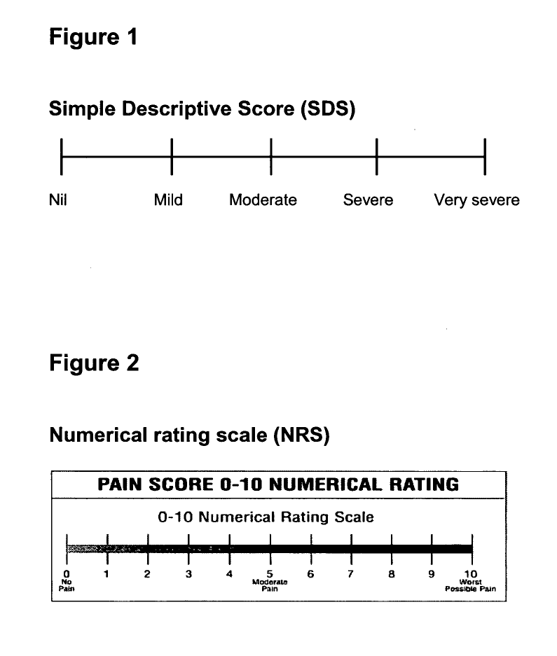

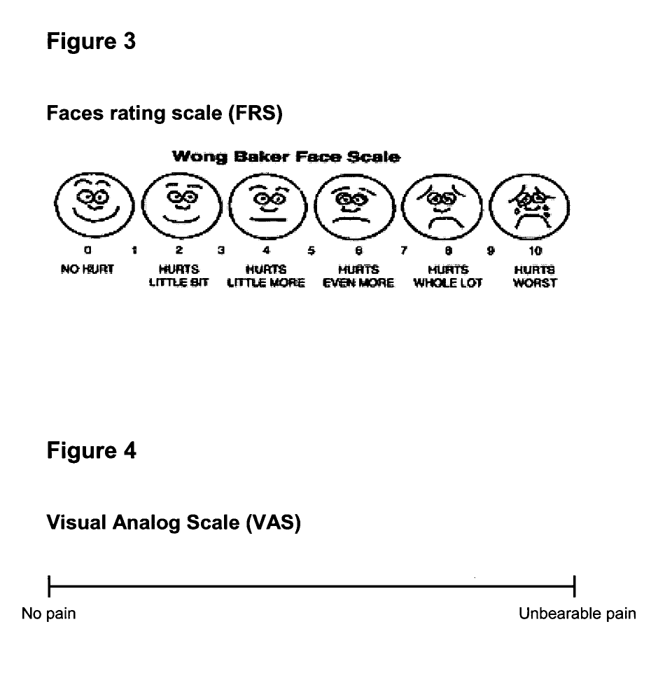

To determine the Intensity rating, the subject first rates the intensity of each of their pain, heat and cold sensations on a scale. This measurement may be done on a suitable scale e.g. a Simple Descriptive Score (SDS); a Visual Analog Scale (VAS); a pictorial scale, such as the Wong-Baker Faces Pain Rating Scale (FRS) (Baker-Lefkowicz et al., 1996); a Numerical Rating Scale (NRS). Most preferably, the intensity is measured on an electronic 10 cm VAS scale. Examples of suitable scales are provided in FIGS. 1-4.

Preferably, the determination of various sensation intensities is carried out by means of a Visual Analog Scale score (or VAS score), e.g. a pain VAS score, a heat VAS score and a cold VAS score. The VAS score may be used in either of the following methods to determine the intensity score for part (c):
(a) used directly [Method 1]; or
(b) further processed by comparing each VAS score to Table 3 below and determining an Allocated score depending on the value range the VAS score falls within [Method 2].

Thus, if for example, Method 1 were used and Subject A gave the pain intensity a VAS score of 6.4 on a scale of 1-10, the pain VAS score would be 6.4.

In contrast, if for example, Method 2 was used and Subject A gave the pain intensity a VAS score of 6.4 on a scale of 1-10, this would generate a pain Allocated score of 5.

TABLE 3
VAS Intensity Score Categories

| Pain | | Heat | | Cold | |
|---|---|---|---|---|---|
| | Allocated | Subject | Allocated | | Allocated |
| Subject score | score | score | score | Subject score | score |
| 0 | 0 | 0 | 0 | 0 | 0 |
| >0-1.5 | 1 | >0-1 | 1 | >0-4 | 1 |
| 1.5-4 | 3 | 1-3 | 3 | 4-6 | 3 |
| >4 | 5 | >3 | 5 | >6 | 5 |

The cold VAS score or cold Allocated score is then divided by two.

The pain VAS score, heat VAS score and half the cold VAS score or the pain Allocated score, heat Allocated score and half the cold Allocated score are then tallied to give an Intensity rating.

Cold Pain Score

The final Descriptor rating and Intensity rating are then tallied to give an overall Cold Pain score. This Cold Pain score is then reviewed to determine if it falls within the category of Normal, Borderline or Abnormal.

Preferably, if Method 1 is used, the Cold Pain scores for Normal subjects are between 0 and 12, more preferably between 0 and 14, most preferably between 0 and 16; and the Abnormal Cold Pain scores are between 18 and 30, more preferably between 17 and 30, most preferably between 16 and 30. If a Borderline score category was desired, it would fall between the upper Normal and lower Abnormal values. Thus, if a subject has a Cold Pain score which falls within the Abnormal category, they would be at increased risk of developing a chronic pain disorder compared with a subject whose Cold Pain score falls within the Normal category.

Preferably, if Method 2 is used, the Cold Pain scores for Normal subjects are between 0 and 10, more preferably between 0 and 11, most preferably between 0 and 12; and the Abnormal Cold Pain scores are between 14 and 20, more preferably between 13 and 20, most preferably between 12 and 20. If a Borderline score category was desired, it would fall between the upper Normal and lower Abnormal values. As above, if a subject has a Cold Pain score which falls within the Abnormal category, they would be at increased risk of developing a chronic pain disorder compared with a subject whose Cold Pain score falls within the Normal category.

Most preferably, this evaluation is based on the values given in Table 4.

TABLE 4
Cold Pain score

| | | Normal | Borderline | Abnormal |
|---|---|---|---|---|
| Method 1 | Descriptor rating + | 0-15 | 16 | 17-30 |
| | Intensity rating | | | |
| Method 2 | Descriptor rating + | 0-11 | 12 | 13-20 |
| | Intensity rating | | | |

It is proposed that a subject with a Cold Pain score within the Abnormal range given in Table 4 above would be at a greater risk of developing a severe chronic pain disorder than a subject with a Cold Pain score in the Normal range.

Examples of data generated using these methods are provided in the Examples section.

The dosage of menthol or a functional equivalent thereof applied topically is preferably applied as a gel application comprising 10-30% menthol.

For the purposes of the present invention, a chronic pain disorder may be defined as pain associated with a chronic disorder, or pain that persists beyond resolution of an underlying disorder or healing of an injury, and that is often more intense than the underlying process would predict. Chronic pain can also be pain persisting for more than one month beyond the resolution of acute tissue injury, pain persisting or recurring for more than three months, or pain associated with tissue injury that is expected to continue or progress (Merck Manual, 2005).

The pain disorder may be varied and includes: musculoskeletal conditions such as whiplash, tennis elbow, osteoarthritis, low back pain, headache and recurrent musculo-tendinous injuries and fibromyalgia. Pain disorders originating from neuropathic conditions may also be included, such as chronic regional pain syndrome, post-herpetic neuralgia and trigeminal neuralgia.

The present invention further provides a method for determining the severity of a pain disorder in a subject, comprising the steps of:
(i) topically applying an effective amount of menthol or a functional equivalent thereof to the subject's painful area and other locations; and
(ii) measuring the response of the subject.

The severity of the pain disorder may be demonstrated by chronicity. Thus, the present invention also provides a method for identifying a chronic pain disorder in a subject comprising the steps of:
(i) topically applying an effective amount of menthol or a functional equivalent thereof to the subject's painful area and other locations; and
(ii) measuring the response of the subject.
   Methods for Assessing Disease Progression and/or Developing and Assessing Efficacy of Treatments

The ability of the method of the present invention to predict the development of a chronic pain disorder and/or assess the severity of a pain disorder renders it useful for assessing the severity of a given pain disorder over time.

In a third aspect, the invention is a method for assessing the severity of a pain disorder over time comprising the repeated steps of:

(i) topically applying an effective amount of menthol or a functional equivalent thereof to the subject's painful area and other locations at least twice over a specific period of time; and

(ii) measuring and comparing the responses of the subject from each application.

In an embodiment of the third aspect of the invention, the invention is a method for assessing the severity of a pain disorder over time, comprising the steps of:
(i) topically applying a composition comprising 5-50% menthol or a functional equivalent thereof to the subject;
(ii) identifying the subject's response to the composition;
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder; and
(iv) repeating steps (I) to (iii) at periodic intervals to develop time course profile of the subject's pain disorder.

It has been found that, as a chronic pain disorder resolves and the subject returns to health, the abnormal response to cold may also be reduced or may disappear. Thus, the present invention may be used over the course of treatment of a chronic pain disorder to assist in the assessment of recovery and healing. It is anticipated that the subject's pain response to the menthol composition may be altered at each time point over the time course if the subject's chronic pain disorder altered. For example, if the subject's chronic pain disorder is healing or being reduced in severity, then the subject's pain response to the menthol stimulus may also be reduced.

The time course over which the method of the present invention is practised can be as short as a few days or a week, or as long as many years. The time points at which the subject's response is measured may be daily, month or yearly. For example, the subject's response to the menthol or functional equivalent thereof may be measured every month for as long as the subject has either an abnormal response to the stimulus, or the subject has the chronic pain disorder. In a further example, the subject's response may be measured weekly for several months upon commencement of a treatment regime, then monthly after that, to measure the ongoing progression of the pain disorder and the ongoing efficacy of the treatment.

The method may also be used to assess the efficacy of treatments for chronic pain disorders.

In a fourth aspect, the invention is a method for assessing the efficacy of a treatment for a subject's pain disorder, comprising the steps of:
(iii) topically applying an effective amount of menthol or a functional equivalent thereof to the subject's painful area and other locations before and after the treatment; and
(iv) measuring and comparing the responses obtained from each application.

In an embodiment of the fourth aspect of the invention, the invention is a method for assessing the efficacy of treatment of a subject's pain disorder, comprising the steps of:
(i) topically applying a composition comprising 5-50% menthol or a functional equivalent thereof to the subject at two different time points, wherein at least one of the selected time points is after the subject receives treatment for the pain disorder;
(ii) identifying the subject's response to the composition at the different time points;
(iii) comparing the subject's response at each time point to a pre-determined set of standard responses to determine the subject's pain threshold, wherein the standard differentiates abnormal menthol responses from normal menthol responses and abnormal menthol responses are indicative of the development of a pain disorder.

In a fifth aspect of the invention, the invention is a method for developing a treatment regime to treat a subject's pain disorder, comprising the steps of:
(i) topically applying a composition comprising 5-50% menthol or a functional equivalent thereof to the subject at two different time points, wherein at least one of the selected time points is after the subject receives treatment for the pain disorder;
(ii) identifying the subject's response to the composition at the different time points;
(iii) comparing the subjects response at each time point to a pre-determined set of standard responses to determine the subject's pain threshold, wherein the standard differentiates abnormal menthol responses from normal menthol responses and abnormal menthol responses are indicative of the development of a pain disorder.

Ideally, the fourth and fifth aspects of the invention are repeated during the course of a subject's treatment for a pain disorder. Such ongoing measurement will provide a time course measure of the subject's pain threshold which can then be related back to the treatment employed in the subject.

As discussed above, it is anticipated that treatments which are effective in healing or reduced the severity of the subject's chronic pain disorder will also reduce the pain response generated by the topical application of a composition comprising 5-50% menthol or a functional equivalent thereof. Thus, the effectiveness of a treatment for a chronic pain disorder may be assessed by the way in which it also affects the subject's response to topical menthol.

A sixth aspect of the invention provides a method for screening a subject to determine if the subject is susceptible to the development of a chronic pain disorder if an injury occurs, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder.

The method of the invention can thus be used to screen individuals or populations to identify individuals who may be at risk of developing chronic pain disorders in the future if they have an injury or develop a disease or condition such as osteoarthritis at some later time point. Such individuals can then be treated appropriately at the time of injury or disease onset to prevent development of a chronic pain disorder.

### Menthol Formulation and Application

Menthol has the following general structure:

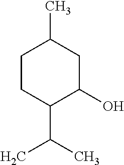

Functional equivalents of menthol according to the present invention include other compounds with the ability to elicit a pain response in a subject with hyperalgesia, such as cold hyperalgesia, but not in a normal or control subject. Functional equivalents may also include compounds that can activate cold receptors such as the TRPM8 receptor and impart signalling in the same or a similar fashion to menthol. Examples of functional equivalents include cinnemaldehyde, icilin, and recently synthesised compounds such as WS-3 (N-ethyl-p-menthane-3-carboxamide), WS-5 (ethyl-3-(p-menthane-3-carboxamido)acetate) and WS-23 (2-isopropyl-N,2,3-trimethylbutyramide) (Leffingwell 2006: http://www.leffingWell.com/cooler_than_menthol). The pain response may be dose dependent. In the present specification, the term “menthol” used alone may be taken to be interchangeable with the term “menthol or a functional equivalent thereof”.

The menthol may be applied at any convenient site including at the site of the pain. For the purposes of the present invention the phrase “at the site of the pain” includes sites proximal to the site of pain that are still capable of eliciting a pain response indicative of the severity of the pain disorder. In this regard, it will be appreciated that the menthol or functional equivalent thereof need not be applied specifically at the site of pain. The menthol or a functional equivalent thereof may be topically administered to any portion of the subject's skin that is suitable and convenient for application. Preferably, the menthol or a functional equivalent thereof is topically applied to the arm or leg of the subject, regardless of where the pain disorder is located. Thus, for example, the subject may be suffering from a chronic pain disorder involving the neck, and the method of the present invention is carried out by topically administering the menthol or a functional equivalent thereof to the skin of the subject's arm.

However, in the early stages of the development of a subject's chronic pain disorder, it may be appropriate to apply the topical menthol or functional equivalent at a site proximal to the site of the pain. For example, if the subject is developing osteoarthritis of the knee and may be at risk of developing a chronic pain disorder, the method of the invention may be carried out by topically applying the menthol of functional equivalent thereof to the skin of the subject's hip.

The menthol or functional equivalent thereof may be applied topically in a number of ways. The menthol may be applied directly, alone, or as part of a composition or formulation such as by painting, wiping, dabbing or spraying. Alternatively, topical administration may be achieved by contacting the site with a means for dispensing an effective amount of the menthol or functional equivalent thereof. Suitable dispensing means are described more fully hereunder. Preferably, the menthol or functional equivalent thereof is topically applied in the form of a gel. However, skin patches, sprays and creams may also be used for topical application.

Menthol compositions and formulations of the present invention include non-sprayable viscous, semi-solid or solid forms that can include a carrier compatible with topical application and have a dynamic viscosity preferably greater than water.

The compositions may contain formulation materials for modifying, maintaining or preserving, for example, the pH, osmolarity, viscosity, clarity, colour, isotonicity, odour, sterility, stability, rate of dissolution or release, adsorption or penetration of the composition. Suitable formulation materials include, but are not limited to antimicrobials; antioxidants (such as ascorbic acid, sodium sulfite or sodium hydrogen-sulfite); buffers (such borate, bicarbonate, Tris-HCl, citrates, phosphates or other organic acids); chelating agents (such as ethylenediamine tetraacetic acid (EDTA)); complexing agents (such as caffeine, polyvinylpyrrolidone, beta-cyclodextrin or hydroxypropyl-beta-cyclodextrin), emulsifying agents; hydrophilic polymers and copolymers (such polyvinylpyrrolidone, hydroxypropylmethylcellulose, hydroxypropylcellulose, carbomers); pressure sensitive adhesives (such as acrylic copolymers); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as water, ethanol, glycerin, polyvinyl alcohol, propylene glycol or polyethylene glycol); surfactants or wetting agents (such as pluronics, sorbitan esters, polysorbates such as polysorbate 20, polysorbate 80, triton, tromethamine, lecithin, cholesterol, tyloxapol); stability enhancing agents; tonicity enhancing agents (such as alkali metal halides, preferably sodium or potassium chloride), delivery vehicles, diluents, excipients and/or pharmaceutical adjuvants including alcohols such as ethanol; release liners and backing membranes (as available for transdermal patch fabrication).

Furthermore, the formulation may comprise a thermo-reversible agent, for example a poloxamer, such that the composition comprising menthol or a functional equivalent thereof may be applied to the subject's skin as a spray or liquid or gel and then will become more viscous as the warmth of the subject's skin heats the composition.

Suitable formulations are well known to those skilled in the art and include, but are not limited to, solutions, suspensions, emulsions, creams, gels, ointments, liniments, salves or aerosols. Particularly preferred topical formulations include ointments, creams, gels and patches.

Ointments generally are prepared using either (1) an oleaginous base, i.e., one consisting of fixed oils or hydrocarbons, such as white petroleum or mineral oil, or (2) an absorbent base, i.e., one consisting of an anhydrous substance or substances which can absorb water, for example anhydrous lanolin. Customarily, following formation of the base, whether oleaginous or absorbent, the active ingredient is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulphate. During formation of the emulsion, menthol can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. The menthol can be added to the formulation at the desired concentration at a point preceding addition of the gelling agent.

The effective amount of the menthol may be varied provided it is an amount that is capable of eliciting a response from a subject that is prognostic and/or indicative of a chronic pain disorder and does not cause undue side effects such as excessive skin reactions. The effective amount may also vary depending on the particular pain disorder and/or the level of chronicity. The effective amount of menthol may be a concentration of 5-50%, 15-45%, 20-40%, 25-35% or 27.5-32.5%. In one particular form of the invention the menthol is used at a concentration of about 30%. More preferably, the effective amount is 10-20%, most preferably 20%.

The pain response of the subject to the menthol or functional equivalent thereof may be measured in various ways. For example, the response may be measured using both quantitative and qualitative measures. The quantitative measure may be a measure of intensity on a suitable numeric scale e.g. a rating of 1-10 on a visual analogue scale, or on a pictorial scale, such as the Wong-Baker Faces Pain Rating Scale. An example of a qualitative measure is a measure of pain quality, such as in the Modified McGill Pain Questionnaire (short form).

### Device for Determining the Severity of a Pain Disorder

The present invention also provides a dispensing means for topically delivering a composition comprising 5-50% menthol or a functional equivalent thereof to a subject in order to determine if they are at risk of developing a chronic pain disorder, the dispensing means comprising the effective amount of menthol or a functional equivalent thereof.

The present invention further provides a dispensing means for topically delivering an effective amount of menthol or a functional equivalent thereof to a subject with a pain disorder, the dispensing means comprising the effective amount of menthol or a functional equivalent thereof.

The dispensing means may deliver the active directly to the subject and thus the delivery of the active may simply be a function of its rate of diffusion from its carrier to the subject, wherein the carrier has no specific controlled release properties. Examples of this form of dispensing means include bandages, cotton wool, gauze and other carriers impregnated or otherwise infused with the active. The dispensing means may also be in the form of a slab of solid or semi-solid gel formulation comprising the menthol, the dispensing means further comprising an occlusive membrane dressing with an adhesive border. Once applied, the solid or semi-solid gel formulation preferably melts or at least becomes more liquid as a result of being heated by the skin of the subject to which it is applied.

Alternatively, the dispensing means may be adapted to deliver the active in a controlled fashion. Thus, the present invention also provides a dispensing means for topically delivering an effective amount of menthol or a functional equivalent thereof to a subject with a pain disorder, the dispensing means comprising the effective amount of menthol or a functional equivalent thereof and a controlled release means.

The controlled release means may be a membrane that, when the dispensing means is in use, is disposed between the active and the intended site of delivery. Thus, the present invention also provides a dispensing means for topically delivering an effective amount of menthol or a functional equivalent thereof to a subject with a pain disorder, the dispensing means comprising (i) the effective amount of menthol or a functional equivalent thereof, (ii) a membrane that, when the dispensing means is in use, is located between the menthol and the intended site of application.

The membrane may be varied and includes: microporous polypropylene and ethyl-vinyl acetate copolymers.

The controlled release means may also be a carrier that is combined or otherwise mixed with the active to control its release.

Thus, the present invention also provides a dispensing means for topically delivering a composition comprising 5-50% menthol or a functional equivalent thereof to a subject in order to determine if they are at risk of developing a chronic pain disorder, the dispensing means comprising (i) the effective amount of menthol or a functional equivalent thereof dispersed or otherwise combined with a controlled release carrier.

The present invention further provides a dispensing means for topically delivering an effective amount of menthol or a functional equivalent thereof to a subject with a pain disorder, the dispensing means comprising (I) the effective amount of menthol or a functional equivalent thereof dispersed or otherwise combined with a controlled release carrier.

The controlled release carrier includes polymers, co-polymers and solvents such as acrylates, ethylene vinyl acetate, ethanol, propylene glycol, glycerin and polyvinyl alcohol.

Preferably, the dispensing means is adapted to be removably fixed to the site of application. The means for removably fixing the dispensing means may also be varied and includes an adhesive member provided on at least a part of the dispensing means. Preferably, the adhesive member and the controlled release member are provided integrally in that the controlled release carrier may also have characteristics that enable it to adhere to the skin of the subject.

### Topical Formulations

The present invention also provides a composition comprising 5-50% menthol or a functional equivalent thereof adapted for topical administration to a subject. In general, any liquid, cream, or gel, or similar substance that does not appreciably react with the menthol or any other of the active ingredients that may be introduced into the composition and which is non-irritating is suitable. As indicated above suitable formulations are well known to those skilled in the art and include, but are not limited to, solutions, suspensions, emulsions, creams, gels, ointments, powders, liniments, salves, and aerosols.

Thus, the present invention also provides a method of producing a menthol composition adapted for topical administration comprising the step of dissolving or combining the menthol in an aqueous or non-aqueous topical pharmaceutically acceptable carrier.

The composition of the present invention may further comprise an auxiliary agent such as any one or more of: preservatives stabilizers, emulsifiers, wetting agents and thickeners such as polymers and copolymers.

### Kits

The methods of the present invention may be carried out using a kit. Thus the present invention provides a kit comprising:
(i) a dispensing means for topically delivering a composition comprising 10-50% menthol or a functional equivalent thereof to a subject at risk of developing a chronic pain disorder, and
(ii) a means for the quantitative and/or qualitative measurement of the subject's response and interpretation thereof.

The present invention also provides a kit comprising:
(i) a dispensing means for topically delivering a composition comprising 10-50% menthol or a functional equivalent thereof to a subject with a chronic pain disorder, and
(ii) a means for the quantitative and/or qualitative measurement of the subject's response and interpretation thereof.

The present invention also provides a kit comprising:
(i) a dispensing means for topically delivering an effective amount of menthol or a functional equivalent thereof to a subject with a pain disorder, and
(ii) a means for the quantitative and/or qualitative measurement of the subject's response and interpretation thereof.

Preferably, the kit further comprises a control dispensing means that includes the same components as the dispensing means, with the exception of the menthol or a functional equivalent thereof. This dispensing means may be used as a control to determine if the mere application of a topical composition is sufficient to generate a pain response in a subject. The application of the control dispensing means can therefore act as a “base line” measurement in a given subject, to which the pain generated by the application of the menthol composition can be compared.

The dispensing means may be varied and are described in more detail elsewhere herein. Preferably the dispensing means is an adhesive patch. More preferably, the dispensing means is a gel.

The kit may further comprise a means for recording the quantitative and/or qualitative measurements. These recording means may be varied and include physical recording means such as paper or electronic recording means such as a computer, palm device or other electronic data storage device. Preferably, the means for recording the measurements is suitable for measuring the Cold Pain score of the subject as described above. Thus, the means for recording may preferably be an electronic 10 cm VAS scale and an interactive electronic data storage device comprising the subject matter of Table 1.

The kit may also further comprise a means for interpreting the measurements. This could be in physical form such as a scale or other data sheet that distinguishes between normal or control responses and abnormal responses indicative of a severe chronic pain disorder. When the kit includes an electronic recording means said means may include software or have the ability to transfer the measurement data to a computer with software that interprets the data and/or applies algorithms thereto to generate a report providing comments on the measurements that may provide further information on the status of the subject's pain disorder.

### ILLUSTRATIONS OF THE INVENTION

### Example 1

### A Matrix/Adhesive Patch

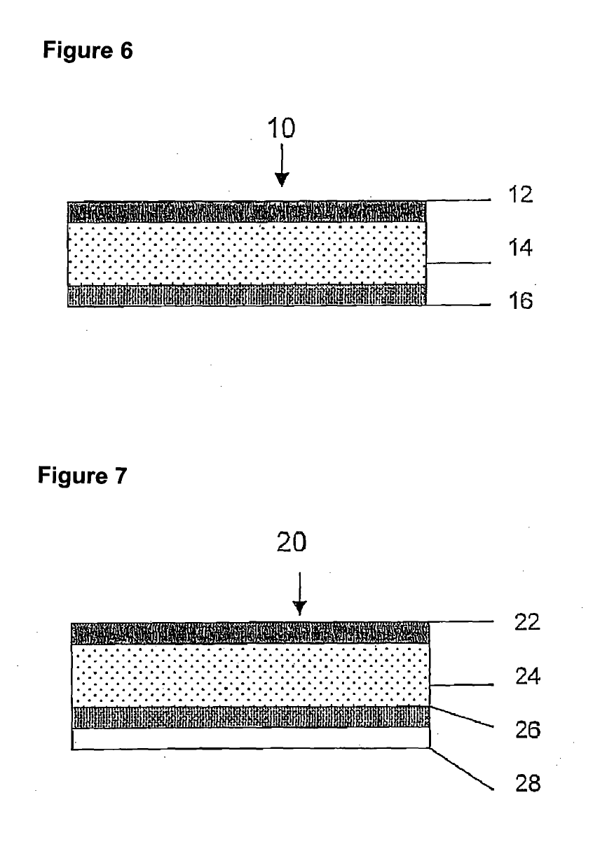

FIG. 6 depicts a dispensing means according to one embodiment of the present invention in the form of a matrix/adhesive patch 10. The patch 10 includes a backing membrane 12 such as polyolefins, elastomers, polyethylene or polyesters, a bioadhesive matrix 14 incorporating menthol and a release liner 16 such as polyethylene or polyesters which may be coated with silicone or fluorocarbons.

In use the patch, containing a predetermined amount of menthol or functional equivalent thereof is affixed to a subject and is delivered through the release liner to the subject's skin.

### Example 2

### A Reservoir/Membrane Patch

FIG. 7 depicts a dispensing means according to another embodiment of the present invention in the form of a reservoir/membrane patch 20. The patch 20 includes a backing membrane 22 such as polyolefins, elastomers, polyethylene or polyesters, a drug reservoir 24 containing menthol or functional equivalent, a rate controlling membrane 26 adapted to release the menthol or functional equivalent at a predetermined rate and an adhesive 28 such as an acrylate to affix the patch to the subjects skin.

In use the patch, containing a predetermined amount of menthol or functional equivalent is affixed to a subject and is delivered through the controlled release membrane to the subject's skin.

### Example 3

### Transdermal Bioadhesive Film

A dispensing means according to another embodiment of the present invention in the form of a transdermal bioadhesive film. The bioadhesive film is a monolayer system that acts as a water permeable transdermal patch on application to the skin. It includes the functions of backing layer, adhesive and drug reservoir in one layer.

### Example 4

### Kit and Method for Determining the Severity of a Whiplash Injury

A kit according to a preferred embodiment of the present invention comprises: (i) a dispensing means in the form of a first patch. incorporating a menthol composition, such as those described in Example 1, 2 or 3 or functional equivalent; (ii) a second adhesive patch with the same appearance as the first patch and incorporating the same composition as the first patch but without menthol or functional equivalent; and (iii) a means for the quantitative and/or qualitative measurement of the subject's response and interpretation thereof.

In use, each patch is affixed to a subject suffering from whiplash at a site where pain is being experienced for a period of about 5 minutes and the intensity and quality of the subject's responses to the first and second patches are recorded and used to determine the severity of the pain disorder.

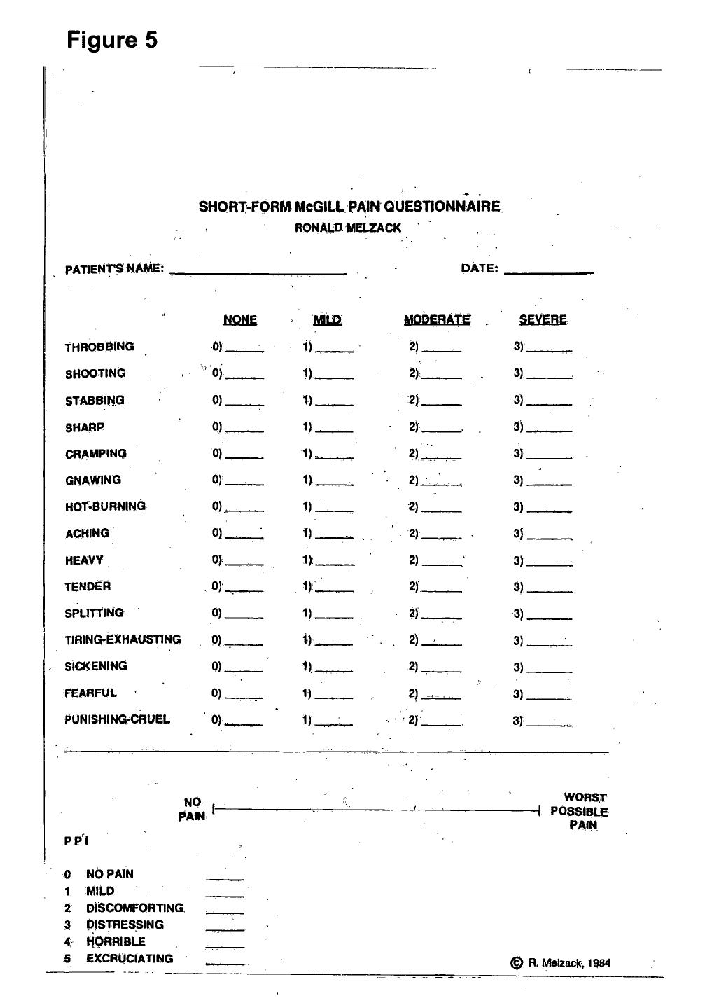

FIG. 5 depicts one embodiment of a means for the quantitative and/or qualitative measurement of the subject's response and interpretation thereof in the form of a recording sheet that, when filled out, enables a practitioner to distinguish between normal responses and abnormal responses indicative of a severe chronic pain disorder.

### Example 5

### Specific Examples of Use of a Cold Pain Test Kit (CPTK) on Subjects

Subjects were tested by topically applying a composition comprising 20% menthol in a gel formulation to their inner forearm. The gel was then covered by an occlusive dressing. The composition was left in place for 15 minutes, during which time the subject was asked every 30 seconds to rate the intensity of the pain, cold and heat sensations they were feeling using 3 electronic 10 cm visual analogue scales (VAS) (potentiometers calibrated to 10 cm each). In addition, every 1 minute, the subject was asked to select a word or words (no limit) from a pre-determined list (modified McGill Pain Scale) to best describe what they were feeling. VAS intensity scores and descriptors were then combined to determine the Cold Pain Score for each individual.

1. DH: subject with history of whiplash and ongoing pain—CPTK Results:
Intensity ratings:
   VAS Pain: 7/10
   VAS Cold: 0/10
   VAS Heat: 6/10
Descriptor Choice Stinging, burning, intense
Classification: Method 1: 17.5/20 Abnormal
   Method 2: 14.5/30 Abnormal
      2. FL: subject with chronic pain from hip osteoarthritis—CPTK results:
Intensity ratings:
   VAS Pain: 4/10
   VAS Cold: 2/10
   VAS Heat: 8/10
Descriptor Choice Tingling, stinging, aching, freezing
Classification: Method 1: 12.75/20 Abnormal
   Method 2: 17.75/30 Abnormal
      3. EL: subject with moderate-severe pain from knee osteoarthritis—CPTK results:
Intensity ratings:
   VAS Pain: 6/10
   VAS Cold: 1/10
   VAS Heat: 8/10
Descriptor Choice Cold, burning
Classification: Method 1: 14.5/20 Abnormal
   Method 2: 18.5/30 Abnormal
      4. SR: subject with knee osteoarthritis but only mild symptoms—CPTK results:
Intensity ratings:
   VAS Pain: 2/10
   VAS Cold: 6/10
   VAS Heat: 0/10
Descriptor Choice: Cold, tingling
Classification: Method 1: 8.5/20 Normal
   Method 2: 10/30 Normal
      5. MM: subject without pain—CPTK Results:
Intensity ratings:
   VAS Pain: 2/10
   VAS Cold: 3/10
   VAS Heat: 0/10
Descriptor Choice Cold, tingling, prickling
Classification: Method 1: 5.7/20 Normal
   Method 2: 6.7/30 Normal

From the data above, it can be seen that normal subjects (5. MM) can be distinguished from those suffering a chronic pain disorder (1. DH; 2. FL). The method of the present invention also distinguishes subjects with pathology but no chronic pain disorder (4. SR) from those with similar levels of pathology who have developed a chronic pain disorder (3. EL).

### Example 6

### Group Data

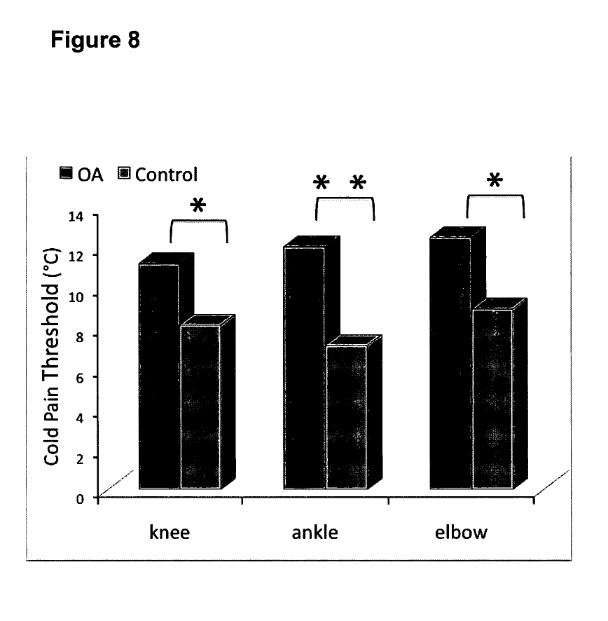

In a study comparing cold responses between subjects with knee osteoarthritis (OA) and matched healthy subjects, there was a significant difference between groups at the OA knee joint, but also at the unaffected ankle and elbow. At each site, the OA subjects showed significantly greater cold pain sensitivity than their healthy counterparts, as shown in FIG. 8.

In a further study, comparing the intensity and descriptor responses of people with hip OA to healthy matched counterparts, a clear, although non-significant, difference was found in cold sensitivity between groups. This study then looked more closely at the OA subjects, separating them into those with mild and those with moderate levels of reported pain (WOMAC). A significant difference was found between these two groups, with those experiencing greater symptoms showing cold sensitivity 85% greater than the mild group.

In this same study, when the descriptor responses were analysed, it was found that those subjects with more severe OA symptoms were more likely to choose words such as “freezing”, “burning” and “stinging”, whereas those with less severe symptoms were more likely to choose “cool”, “cold” or “dull”.

These results match other studies of subjects without known pathology, where a clear relationship between intensity of response to a strong cold stimulus correlates to choice of particular descriptors. Those with abnormally high responses (VAS scores of pain >4/10 or cold pain threshold of >15° C.) were likely to choose thermal words such as “icy” or “freezing” and “burning” or “searing” plus more intense dysaesthtic words such as “stinging” and “prickling”.

TABLE 5
Percentage choice of descriptors at cold pain threshold

| Descriptor | % choice |
|---|---|
| Icey | 50.0 |
| Cold | 43.4 |
| Stinging | 27.9 |
| Tingling | 27.6 |
| Prickling | 23.2 |
| Penetrating | 22.4 |

1. A method for determining the severity of a pain disorder in a subject, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder.

2. A method for identifying the development of a chronic pain disorder in a subject, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject has a chronic pain disorder, wherein the standard differentiates abnormal menthol responses from normal menthol responses and abnormal menthol responses are indicative of chronic pain in a subject.

3. A method according to claim 1, wherein the severity of the pain disorder is assessed over time, comprising the steps of:
(i) topically applying a composition comprising 5-50% menthol or a functional equivalent thereof to the subject;
(ii) identifying the subject's response to the composition;
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder; and
(iv) repeating steps (i) to (iii) at periodic intervals to develop time course profile of the subject's pain disorder.

4. A method according to claim 1, wherein the severity of the subject's pain disorder in response to treatment is assessed, comprising the steps of:
(i) topically applying a composition comprising 5-50% menthol or a functional equivalent thereof to the subject at two different time points, wherein at least one of the selected time points is after the subject receives treatment for the pain disorder;
(ii) identifying the subject's response to the composition at the different time points;
(iii) comparing the subject's response at each time point to a pre-determined set of standard responses to determine the subject's pain threshold, wherein the standard differentiates abnormal menthol responses from normal menthol responses and abnormal menthol responses are indicative of the development of a pain disorder.

5. A method according to claim 1, wherein the severity of the subject's pain disorder is assessed during the development or tailoring of a treatment regime to treat a subject's pain disorder, comprising the steps of:
(i) topically applying a composition comprising 5-50% menthol or a functional equivalent thereof to the subject at two different time points, wherein at least one of the selected time points is after the subject receives treatment for the pain disorder;
(ii) identifying the subject's response to the composition at the different time points;
(iii) comparing the subject's response at each time point to a pre-determined set of standard responses to determine the subject's pain threshold, wherein the standard differentiates abnormal menthol responses from normal menthol responses and abnormal menthol responses are indicative of the development of a pain disorder.

6. A method for screening a subject to determine if the subject is susceptible to the development of chronic pain if that subject is stricken with a pain disorder, comprising the steps of:
(i) topically applying a composition comprising 5-50% menthol or a functional equivalent thereof to the subject;
(ii) identifying the subject's response to the composition; and
(iii) comparing the subject's response to a pre-determined set of standard responses to determine whether the subject is susceptible to the development of a pain disorder, wherein the standard differentiates abnormal menthol responses from normal menthol responses and abnormal menthol responses are indicative of the development of a pain disorder.

7. A method according to claim 1, wherein a subject's menthol response is measured using the Cold Pain Score determined by:
(a) rating the subject's menthol response according to a description of the subject's sensation to the menthol, said description being defined by selecting as many or as few of the words from the following list to describe the sensation(s), the list being: cool, cold, icy, freezing, warm, hot, burning, numb, tingling, itchy, prickling, stinging, dull, sharp, penetrating, intense, aching, pulsing/throbbing, spreading; and
(b) rating the intensity of the subject's cold sensation, heat sensation and pain sensation generated in response to the menthol or functional equivalent thereof on a scale from 1 to 10.

8. The method according to claim 1, wherein the functional equivalents are chosen from the list comprising: cinnemaldehyde, icilin, WS-3 (N-ethyl-p-menthane-3-carboxamide), WS-5 (ethyl-3-(p-menthane-3-carboxamido)acetate) and WS-23 (2-isopropyl-N,2,3-trimethylbutyramide).

9. The method according to claim 1, wherein the menthol or a functional equivalent thereof is topically applied to an arm or leg of the subject.

10. The method according to claim 1, wherein the menthol or a functional equivalent thereof is at a concentration of 5-50%.

11. (canceled)

12. (canceled)

13. A kit comprising:
(i) a dispensing means for topically delivering a composition comprising 5-50% menthol or a functional equivalent thereof to a subject at risk of developing a chronic pain disorder or a subject with a chronic pain disorder; and
(ii) a means for the quantitative and/or qualitative measurement of the subject's response and interpretation thereof.

14. (canceled)

15. The method according to claim 4, wherein a subject's menthol response is measured using the Cold Pain Score determined by:
(a) rating the subject's menthol response according to a description of the subject's sensation to the menthol, said description being defined by selecting as many or as few of the words from the following list to describe the sensation(s), the list being: cool, cold, icy, freezing, warm, hot, burning, numb, tingling, itchy, prickling, stinging, dull, sharp, penetrating, intense, aching, pulsing/throbbing, spreading; and
(b) rating the intensity of the subject's cold sensation, heat sensation and pain sensation generated in response to the menthol or functional equivalent thereof on a scale from 1 to 10.

16. The method according to claim 4, wherein the functional equivalents are chosen from the list comprising: cinnemaldehyde, icilin, WS-3 (N-ethyl-p-menthane-3-carboxamide), WS-5 (ethyl-3-(p-menthane-3-carboxamido) acetate) and WS-23 (2-isopropyl-N,2,3-trimethylbutyramide).

17. The method according to claim 4, wherein the menthol or a functional equivalent thereof is topically applied to an arm or leg of the subject.

18. The method according to claim 4, wherein the menthol or a functional equivalent thereof is at a concentration of 5-50%.

19. The method according to claim 5, wherein a subject's menthol response is measured using the Cold Pain Score determined by:
(c) rating the subject's menthol response according to a description of the subject's sensation to the menthol, said description being defined by selecting as many or as few of the words from the following list to describe the sensation(s), the list being: cool, cold, icy, freezing, warm, hot, burning, numb, tingling, itchy, prickling, stinging, dull, sharp, penetrating, intense, aching, pulsing/throbbing, spreading; and
(d) rating the intensity of the subject's cold sensation, heat sensation and pain sensation generated in response to the menthol or functional equivalent thereof on a scale from 1 to 10.

20. The method according to claim 5, wherein the functional equivalents are chosen from the list comprising: cinnemaldehyde, icilin, WS-3 (N-ethyl-p-menthane-3-carboxamide), WS-5 (ethyl-3-(p-menthane-3-carboxamido) acetate) and WS-23 (2-isopropyl-N,2,3-trimethylbutyramide).

21. The method according to claim 5, wherein the menthol or a functional equivalent thereof is topically applied to an arm or leg of the subject.

22. The method according to claim 5, wherein the menthol or a functional equivalent thereof is at a concentration of 5-50%.
